(19) United States
(12) Patent Application Publication
(10) Pub. No.: US 2013/0217111 A1
(43) Pub. Date: Aug. 22, 2013

(54) Waste Disposal Device

(71) Applicant: Kuang-Nan Chang, Taichung City (TW)

(72) Inventor: Kuang-Nan Chang, Taichung City (TW)

(73) Assignee: TA YA KITCHEN UTENSIL CO., LTD, Taichung City (TW)

(21) Appl. No.: 13/400,892

(22) Filed: Feb. 21, 2012

(51) Int. Cl. C12M 1/10

(52) U.S. Cl. 435/290.1

(57) ABSTRACT

A waste disposal device includes a grinder tank disposed within a housing and having an inner rounded surface angularly extending about an axis for loading of kitchen waste therein, and a grinder assembly rotatably mounted in the tank about the axis to stir and shred the waste and configured to force the waste to advance forward so that the shredding and drying process can be carried out efficiently. A condensing unit is disposed to cool and condense hot air in the housing into water which is drained through a drain port to thereby reduce leakage of unpleasant odor through an access opening of the housing.

### BACKGROUND OF THE INVENTION

1. Field of the Invention

This invention relates to a waste disposal device, more particularly to a waste disposal device for stirring, shredding and drying kitchen waste.

2. Description of the Related Art

A conventional waste disposal device, such as that disclosed in U.S. Pat. Nos. 5,710,042 and 6,620,613, generally includes a grinder tank for loading of kitchen waste thereinto, a rotary shaft having vanes or blades rotatably mounted into the grinder tank to stir and shred the loaded kitchen waste, and a heater disposed to heat and dry the loaded kitchen waste during the stirring and shredding process. In such conventional waste disposal device, unpleasant odor is generated and leaks out during storage of the loaded kitchen waste or the heating process. Besides, as the rotary shaft extends in a horizontal direction, the stirring and shredding effect is unsatisfactory.

### SUMMARY OF THE INVENTION

An object of the present invention is to provide a waste disposal device which can efficiently stir, shred, heat and dry kitchen waste and which prevents odor leakage.

According to this invention, the waste disposal device includes a housing having top and bottom ends which are spaced apart from each other in an upright direction, and a surrounding wall which extends between the top and bottom ends to cooperatively define a housing chamber. The top end has an access opening for loading of kitchen waste thereinto. A grinder tank is disposed in the housing chamber and extends along an axis that is oriented to be inclined relative to the bottom end of the housing. The grinder tank has an inner rounded surface which extends angularly about the axis and in a direction of the axis to terminate at front and rear edges such that the front edge is higher than the rear edge, and front and rear bearing walls which are respectively joined with the front and rear edges to cooperate with the inner rounded surface to define a stirring and shredding subchamber so as to permit loading of the kitchen waste therein. The inner rounded surface has a discharge port disposed adjacent to the front edge. A heating element is disposed to heat the kitchen waste in the stirring and shredding subchamber. A rotary shaft extends into the stirring and shredding subchamber along the axis and is rotatably mounted on the front and rear bearing walls about the axis. A shredding blade unit is disposed on the rotary shaft and has leading and trailing blade segments which are angularly displaced from each other about the axis, and which are opposite to each other in the direction of the axis. Each of the leading and trailing blade segments has a forward-facing major surface oriented to be inclined relative to the axis. By virtue of rotation of the shredding blade unit with the rotary shaft, the kitchen waste is successively forced by the forward-facing major surfaces to advance forward to the discharge port. A drive unit is disposed to drive the rotary shaft to rotate about the axis.

### BRIEF DESCRIPTION OF THE DRAWINGS

Other features and advantages of the present invention will become apparent in the following detailed description of the preferred embodiment of the invention, with reference to the accompanying drawings, in which:

FIG. 1 is a perspective view of an embodiment of a waste disposal device according to this invention;

FIG. 2 is an exploded perspective view of the embodiment;

FIG. 3 is a sectional view taken along line of FIG. 1;

FIG. 4 is a perspective view of a heating element of the embodiment;

FIG. 5 is a perspective view showing a condensing unit in an opened state;

FIG. 6 is a sectional view similar to FIG. 3, showing the embodiment in an operated state;

FIG. 7 is a partly sectioned view showing the structural relationship between the condensing unit and a drain port of a housing; and

FIG. 8 is a sectional view similar to FIG. 3, showing loaded kitchen waste discharged from a grinder tank to a collection bin.

### DETAILED DESCRIPTION OF THE PREFERRED EMBODIMENT

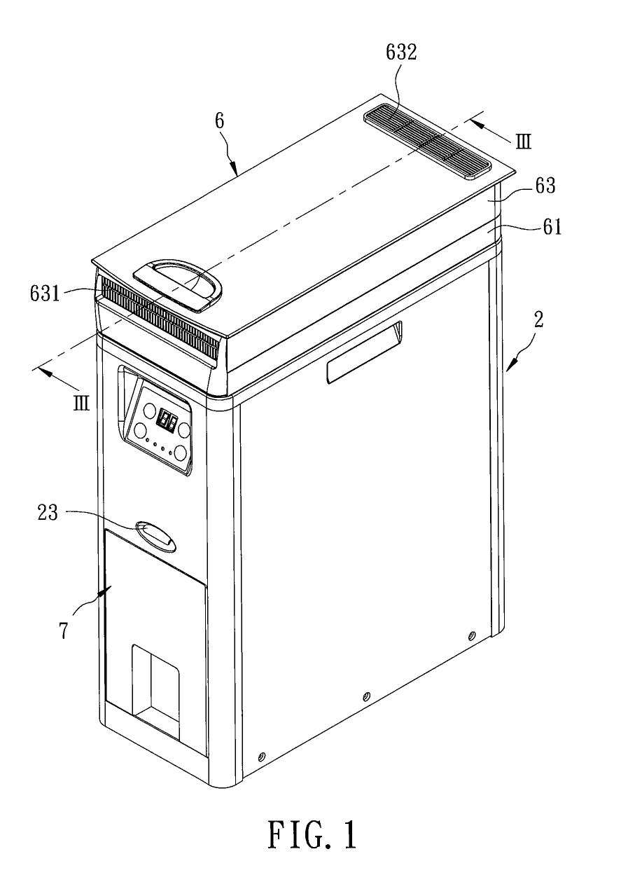

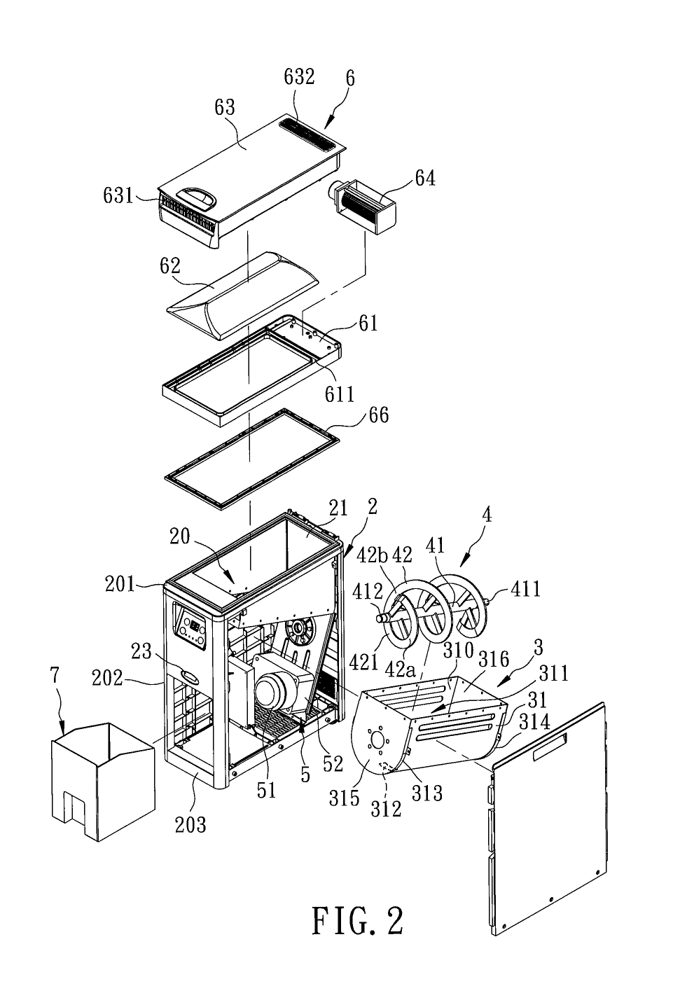

Referring to FIGS. 1 to 3, an embodiment of a waste disposal device according to the present invention is shown to comprise a housing 2, a tank unit 3, a grinder assembly 4, a drive unit 5, a condensing unit 6, and collection bin 7.

The housing 2 has top and bottom ends 201, 203 spaced apart from each other in an upright direction, and a surrounding wall 202 extending between the top and bottom ends 201, 203 so as to cooperatively define a housing chamber 20. The top end 201 has an access opening 21 for loading of kitchen waste thereinto. The surrounding wall 202 has a drain port 22 disposed adjacent to the bottom end 203. The housing 2 further has an operating handle 23 which is movably mounted on a front side thereof.

The tank unit 3 includes a grinder tank 31 which is disposed in the housing chamber 20 and which extends along an axis (L) that is oriented to be inclined relative to the bottom end 203, and a heating element 32 which is disposed on an outer rounded surface of the grinder tank 31.

The grinder tank 31 has an inner rounded surface 310 which extends angularly about the axis (L) and in a direction of the axis (L) to terminate at front and rear edges 313, 314 such that the front edge 313 is higher than the rear edge 314, and front and rear bearing walls 315, 316 which are respectively joined with the front and rear edges 313, 314 to cooperate with the inner rounded surface 310 to define a stirring and shredding subchamber 311 that is communicated with the access opening 21 to permit the kitchen waste to be loaded therein. The inner rounded surface 310 has a discharge port 312 which is disposed adjacent to the front edge 313. Agate 317 is disposed to openably close the discharge port 312. The operating handle 23 is coupled to the gate 313 to control opening of the discharge port 312. The axis (L) and a plane (X) defined by the bottom end 203 include an angle (θ) ranging between 15 and 35 degrees. In this embodiment, the angle (θ) is approximately 20 degrees.

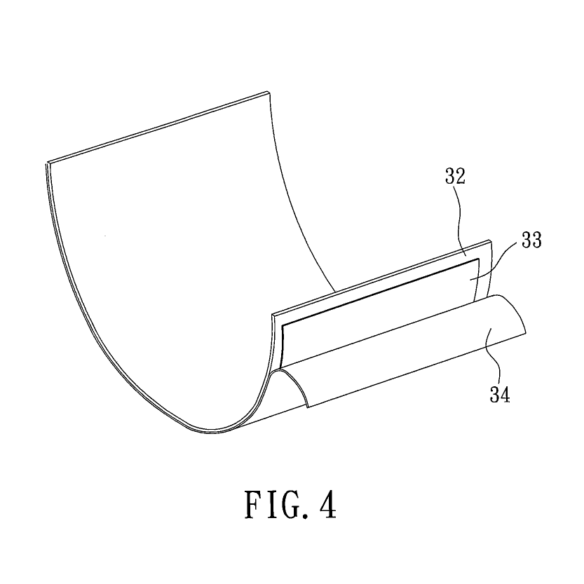

With reference to FIG. 4, the heating element 32 includes a heating sheet 33 which can heat the kitchen waste in the stirring and shredding subchamber 311, and a heat insulating sheet 34 which enhances the heating effect of the heating element 32.

The grinder assembly 4 includes a rotary shaft 41 which extends into the stirring and shredding subchamber 311 along the axis (L), and a shredding blade unit 42 which is disposed on the rotary shaft 41. The rotary shaft 41 has two ends 411, 412 which are respectively mounted on the front and rear bearing walls 315, 316 to permit rotation of the rotary shaft 41 about the axis (L). The shredding blade unit 42 has a plurality of leading blade segments (42a) and a plurality of trailing blade segments (42b) which are angularly displaced from one another about the axis (L), and which are opposite to each other in the direction of the axis (L) to cooperatively define a spiral contour that extends spirally around the axis (L). Each of the leading and trailing blade segments (42a, 42b) has a forward-facing major surface which is oriented to be inclined relative to the axis (L).

The drive unit 5 includes a drive motor 51 mounted below the tank unit 3, a speed-reduction gearbox 52 coupled with the drive motor 51, a transmitting wheel 53 coupled with the end 411 of the rotary shaft 41, and a chain 54 interconnecting the transmitting wheel 53 and the speed-reduction gearbox 52 so as to enable the grinder assembly 4 to be driven by the drive motor 51 to rotate about the axis (L).

The condensing unit 6 has an upper lid half 63 which is configured to cover the access opening 21, a lower lid half 61 which is interposed between the upper lid half 63 and the grinder tank 31 and which has a surrounding trough 611, a condenser member 62 in the form of a condenser plate which is interposed between the upper and lower lid halves 63, 61 such that hot air in the housing chamber 20 is cooled and condensed by the condenser member 62 into water that drips into the surrounding trough 611, and a drain conduit 65 which is disposed on the surrounding wall 202 and which extends in the upright direction to communicate the surrounding trough 611 with the drain port 22. A magnetically closing member 66 is disposed on a periphery of the lower lid half 61 to be magnetically attached to a periphery of the top end 203 of the housing 2 so as to ensure an air-tight engagement between the lower lid half 61 and the housing 2. The magnetically closing member 66 may be a magnet. Further, the upper lid half 63 has an intake port 631 and a vent port 632 communicated with the housing chamber 20, and an air blower 64 disposed to generate an airflow from the intake port 631 to the vent port 632.

The collection bin 7 is disposed in the housing chamber 20 under the discharge port 312, and is in the form of a drawer to be pulled out of the housing 2.

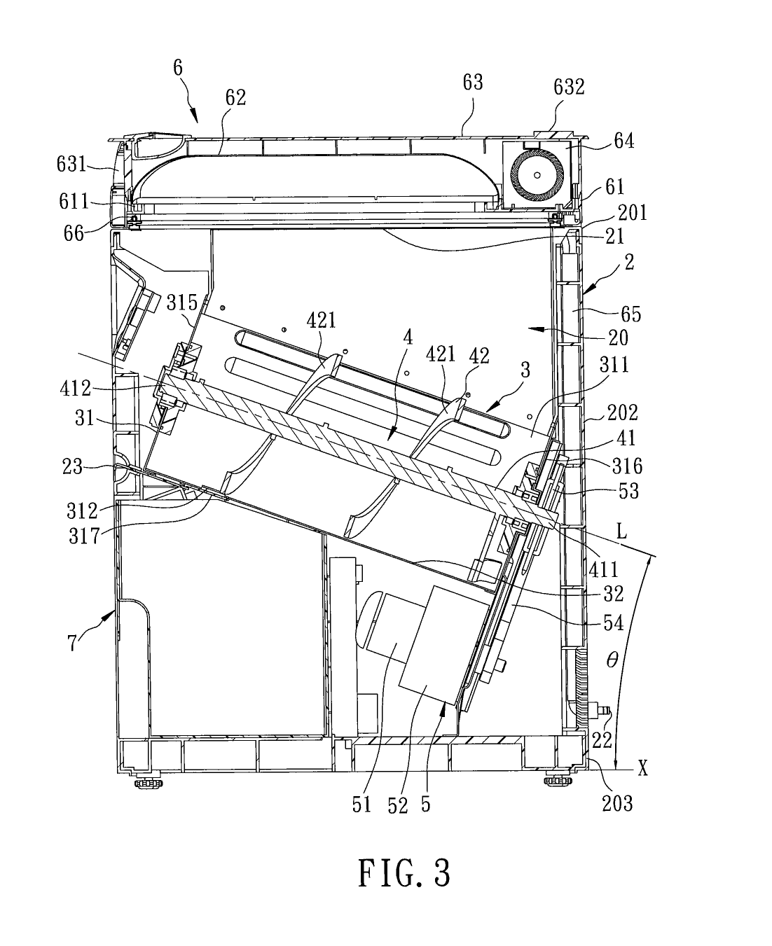

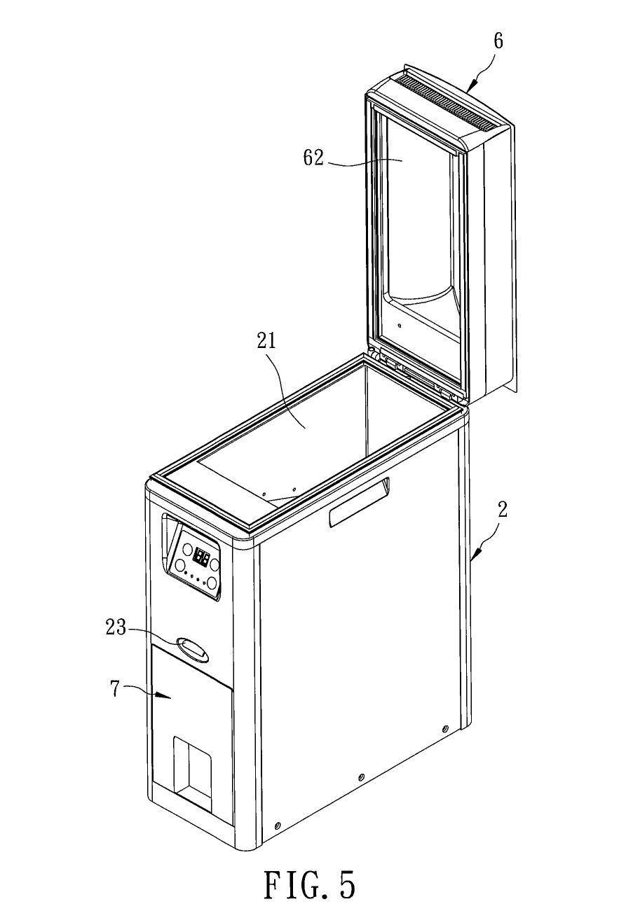

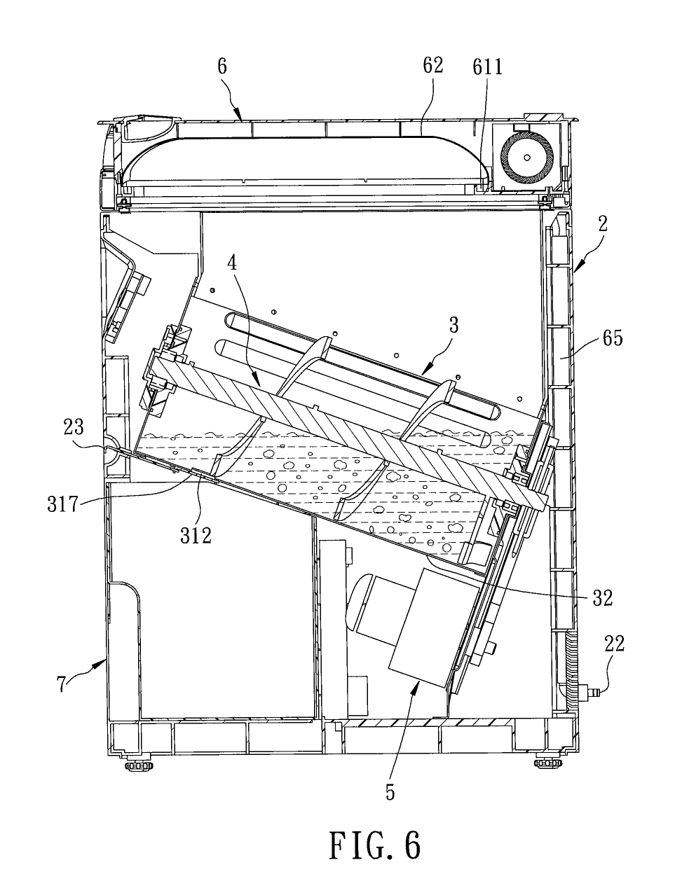

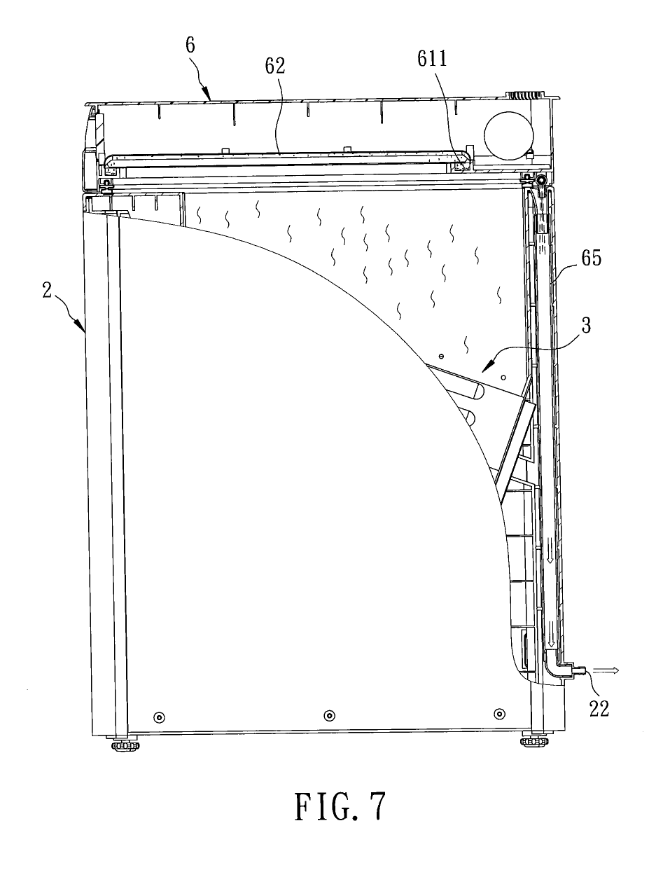

Referring to FIGS. 3 and 5, in use, the condensing unit 6 is moved to an opened state to permit loading of kitchen waste and fermentation bacteria into the stirring and shredding subchamber 311 from the access opening 21. Referring to FIGS. 6 and 7, subsequently, the condensing unit 6 is moved to a closed state, and the drive unit 5 and the heating element 32 are actuated in a known manner to drive the grinder assembly 4 to rotate and to heat the kitchen waste in the stirring and shredding subchamber 311. During the heating-shredding process, the kitchen waste can be stirred and heated to accelerate fermentation, while the hot air is cooled and condensed by the condenser member 62 into water which drips into the surrounding trough 611 to be drained out of the drain port 22. By virtue of the air blower 64 to generate the airflow from the intake port 631 to the vent port 632, the condensing effect of the condenser member 62 can be enhanced.

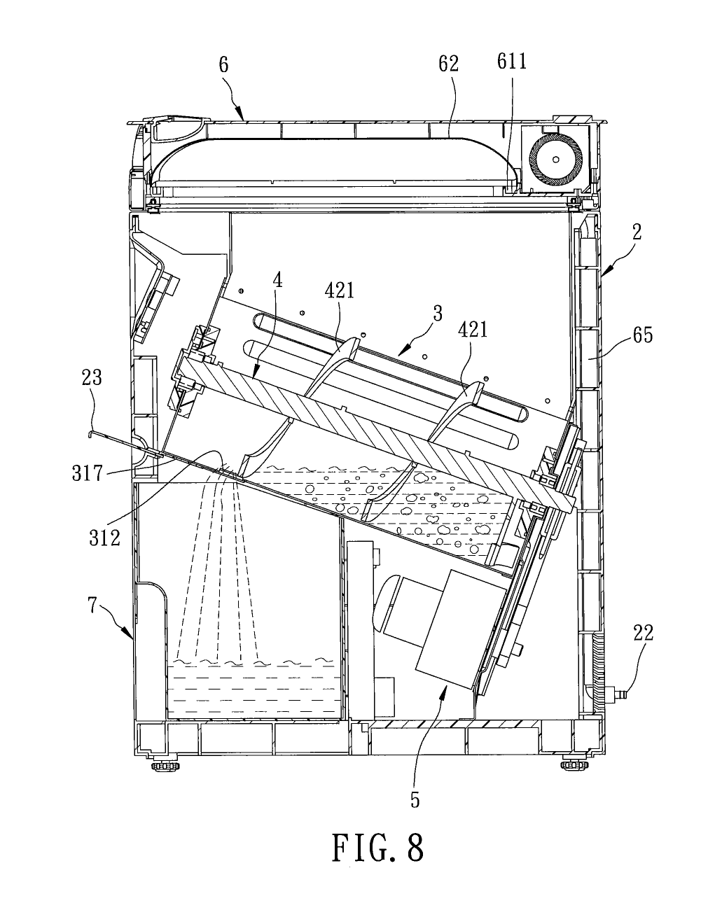

Referring to FIG. 8, when a predetermined amount of waste is loaded, or when the heating-shredding process is completed, the gate 317 is controlled by pulling the operating handle 23 to open the discharge port 312 while the grinder assembly 4 is kept rotating, and the kitchen waste can be successively forced by the forward-facing major surfaces 421 to advance forward so as to be drawn out of the discharge port 312 and collected in the collection bin 7.

As illustrated, by virtue of the grinder assembly 4, the tank unit 3 and the condensing unit 6, the kitchen waste can be evenly stirred and shredded, and moisture in the kitchen waste, after being vaporized, can be condensed to be discharged from the drain port 22, thereby permitting the kitchen waste to be shred and dried efficiently while leakage of odor into the ambient air can be prevented. Further, by virtue of the shredding blade unit 42 which is moved along a spiral path around the axis (L), i.e., in both a circumferential direction and an axial direction, the stirring, shredding and drying process and the fermentation and decomposition of the shredded waste can proceed efficiently. Furthermore, by virtue of the inclined tank unit 3, a large amount of waste can fall down toward a rear region of the stirring and shredding subchamber 311 so as to be successively forced to advance forward by rotation of the shredding blade unit 42, thereby enhancing the stirring and shredding effect. Besides, by virtue of the magnetically closing member 66, an air-tight engagement between the condensing unit 6 and the housing 2 can be ensured, thereby preventing odor leakage.

While the present invention has been described in connection with what is considered the most practical and preferred embodiment, it is understood that this invention is not limited to the disclosed embodiment but is intended to cover various arrangements included within the spirit and scope of the broadest interpretations and equivalent arrangements.

What is claimed is:

1. A waste disposal device comprising:
a housing having top and bottom ends which are spaced apart from each other in an upright direction, and a surrounding wall which extends between said top and bottom ends and cooperates therewith to define a housing chamber, said top end having an access opening for loading of kitchen waste therethrough;
a grinder tank which is disposed in said housing chamber and which extends along an axis that is oriented to be inclined relative to said bottom end, said grinder tank having an inner rounded surface which extends angularly about the axis and in a direction of the axis to terminate at front and rear edges such that said front edge is higher than said rear edge, and front and rear bearing walls which are respectively joined with said front and rear edges to cooperate with said inner rounded surface to define a stirring and shredding subchamber that is communicated with said access opening so as to permit loading of the kitchen waste therein, said inner rounded surface having a discharge port which is disposed adjacent to said front edge;
a heating element disposed to heat the kitchen waste in said stirring and shredding subchamber;
a rotary shaft which extends into said stirring and shredding subchamber along the axis and which is rotatably mounted on said front and rear bearing walls about the axis;
a shredding blade unit disposed on said rotary shaft, and having leading and trailing blade segments which are angularly displaced from each other about the axis, and which are opposite to each other in the direction of the axis, each of said leading and trailing blade segments having a forward-facing major surface which is oriented to be inclined relative to the axis such that, by virtue of rotation of said shredding blade unit with said rotary shaft, the kitchen waste is successively forced by said forward-facing major surfaces of said leading and trailing blade segments to advance forward to said discharge port; and
a drive unit disposed to drive said rotary shaft to rotate about the axis.

2. The waste disposal device as claimed in claim 1, wherein
said surrounding wall has a drain port disposed adjacent to said bottom end,
said device further comprising a condensing unit having
an upper lid half which is configured to cover said access opening,
a lower lid half which is interposed between said upper lid half and said grinder tank and which has a surrounding trough,
a condenser member which is interposed between said upper and lower lid halves such that hot air in said housing chamber is cooled and condensed by said condenser member into water that drips into said surrounding trough, and
a drain conduit which extends in the upright direction to communicate said surrounding trough with said drain port.

3. The waste disposal device as claimed in claim 2, wherein said upper lid half has an intake port and a vent port communicated with said housing chamber, and an air blower disposed to generate an airflow from said intake port to said vent port so as to provide ventilation to said condenser member, thereby enhancing the condensing effect thereof.

4. The waste disposal device as claimed in claim 2, further comprising a magnetically closing member configured to be disposed between peripheries of said lower lid half and said top end so as to ensure an air-tight engagement therebetween.

5. The waste disposal device as claimed in claim 1, further comprising a collection bin disposed in said housing chamber under said discharge port.

6. The waste disposal device as claimed in claim 5, wherein said grinder tank has a gate which is disposed to openably close said discharge port, said housing having an operating handle which is coupled to said gate to control opening of said discharge port.

7. The waste disposal device as claimed in claim 1, wherein the axis and a plane defined by said bottom end include by an angle ranging between 15 and 35 degrees.
